(19) United States
(12) Plant Patent Application Publication
(10) Pub. No.: US 2004/0139521 P1
(43) Pub. Date: Jul. 15, 2004

(54) Verbena plant named 'Darlena'

(76) Inventor: Henricus G.W. Stemkens, Hoorn (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/342,814

(22) Filed: Jan. 15, 2003

(51) Int. Cl. A01H 5/00

(52) U.S. Cl. PLT/308

(57) ABSTRACT

A new and distinct variety of Verbena plant particularly distinguished by its neonrose flowers, early flowering, and a spreading habit that is first semi-erect and later spreading.

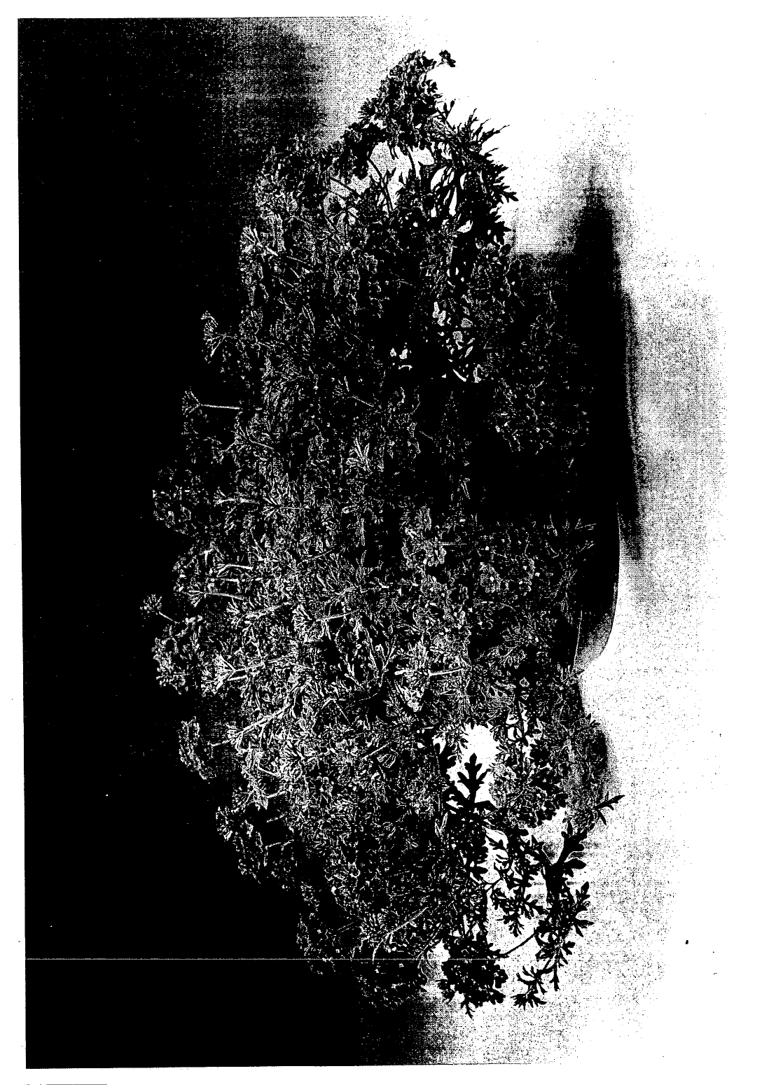

### VARIETAL DENOMINATION

‘Darlena’

### BACKGROUND OF THE NEW PLANT

The present invention comprises a new distinct cultivar of Verbena, botanically known as Verbena×hybrida.

The new cultivar is propagated from cuttings resulting from the cross of ‘W720’ and ‘T593’. ‘W720’ is a rose flowering Verbena having a spreading habit. ‘W720’ is not commercially available and is not known by any synonyms. ‘T593’ is a scarlet flowering Verbena having a semi-erect habit. ‘T593’ is not commercially available and is not known by any synonyms. Neither ‘W720’ nor ‘T593’ has been patented.

As a result of this cross the present cultivar was created in 1999 in Enkhuizen, Netherlands and has been repeatedly asexually reproduced by cuttings in Enkhuizen, Netherlands and Sarrians, France over a three-year period. It has been found to retain its distinctive characteristics through successive propagations, and this novelty appears to be firmly fixed.

‘Darlena’ is closely related to the patented variety Florena, from which it differs in vigor, flower color and length of spikes.

This new Verbena plant is an annual in most climatical zones in the US, only in zones 9 and 10 it is a perennial plant.

### DESCRIPTION OF THE DRAWING

This new Verbena plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Verbena The data which defines these characteristics were collected from asexual reproductions carried out in Enkhuizen, Netherlands. The plant history was taken on 14 weeks old plants, blossomed under natural light in a greenhouse.

Color readings were taken in the greenhouse under ambient light. Color references are primarily to the RHS Colour Chart of The Royal Horticultural Society of London.

Differences between the new cultivar ‘Darlena’ its
parents and a similar cultivar

| | | | | ‘Temari |
|---|---|---|---|---|
| | ‘Darlena’ | ‘W720’ | ‘T593’ | coral rose’ |
| Flower color | Neon rose | Rose | Scarlet | Coral rose |
| Earliness | Early | Very early | Late | Late |
| Seed set | No | No | Much | Abundant |
| Leaf | Double | Double | No | No |
| incision | incised | incised | incisions | incisions |

The plant:

Classification.—Botanical: Verbena×hybrida.

Parentage.—Female parent: A seedling named ‘W720’ is one of our seedlings from our W-generation of plants bred in 1994. — Pollen parent: A seedling named ‘T593’ is one of our seedlings from our T-generation of plants bred in 1991.

Growth habit.—Semi-correct, later spreading.

Plant height.—16-24 cm.

Speading area of plant.—35-65 cm.

Growth rate.—Hanging and vigorous.

Strength.—Resistant to hot and cold weather.

Branching character.—Freely branching and lateral branching at every node.

Blooming period.—From April until November.

The stem:

Diameter.—2-2.5 mm.

Shape.—Tetragonal.

Color.—141C.

Anthocyan pigmentation.—Present.

Length of internode.—25-40 mm, depending on the light where the plant is propagated.

Pubescence.—Pubescence is present.

The foliage:

Phyllotaxis.—Opposite.

Shape of blade.—Broadly ovate.

Texture.—Upper side: Smooth — Lower side: Smooth.

Venation.—Pinnate.

Leaf margin.—Laciniate.

Leaf base.—Hastate.

Leaf apex.—Apiculate.

Length.—16-28 mm.

Width.—14-24 mm.

Depth of incision.—8-12 mm.

Color.—Upper side: 141C — Lower side: 138B.

Pubescence.—Some pubescence is present.

Length of leaf stem.—8-15 mm.

Petiole surface structure.—Slightly pubescent.

The bud:

Peduncle length.—30-40 mm, depending on season.

Size of the bud.—Length: 8-12 mm — Diameter: 2 mm.

Shape.—Elongated and ovate.

Color.—137D.

Sepals.—Color: 138B — Form: Upright — Number: 5, fused — Size: 5 mm — Shape: Elongated.

The flower:

Facing direction.—Upward.

Outward curvature of petal.—Slightly curved.

Diameter.—14-20 mm.

Height.—14-18 mm.

Borne.—In a cluster.

Form.—Salverform; sessile on terminal spikes.

Cluster.—Spike.

Color.—67A, a bit more blue.

Eye.—A very small (1 mm) greenish Eye (155B) is present. Typically three out of the five petals exhibit this greenish coloration.

Overlapping of the petals.—Separate.

Number of petals.—Gamopetalous, five lobed.

Shape of the petals.—Each petal is heart shaped at the apex and grown togheter at the base.

Petal margin.—Entire.

Petal surface texture.—Smooth.

Size of the petal.—Length: 5-7 mm — Width: 6-8 mm.

Calyx length.—8-10 mm.

Antocyan pigmentation of calyx limb.—Absent.

Color of the calyx.—137A.

No. of flowers per spike.—25-35.

Fragrance.—No fragrance.

Lastingness of the bloom.—New florets continue to open in one spike over a period of 14 days.

The reproductive organs:

Androecium.—Stamen quantity: Four — Anther shape: Ovoid — Anther length: 1 mm — Anther color: 144C — Amount of pollen: No pollen.

Gynoecium.—Pistil quantity: 1 — Stigma shape: Bi-lobed — Pistil length: 1.8-2.2 mm — Stigma color: 144C — Style length: 1.6 cm — Style color: 144D — Ovary color: 144C.

The seed:

Seedset.—No seedset has been observed.

The roots:

Type of roots.—Fibrous. Roots starts to grown on every part of the stem That contacts the soil, so not only at the nodes.

Physiological and ecological characteristics: Good tolerance to heat and cold. Strong resistance to pests and diseases, particularly powdery mildew.

What is claimed is:

1. A new and distinct cultivar of Verbena plant named ‘Darlena,’ substantially as illustrated and described herein.
